(19) United States
(12) Patent Application Publication
(10) Pub. No.: US 2026/0108189 A1
(43) Pub. Date: Apr. 23, 2026

(54) MULTI-PARAMETER WEARABLE PATCH FOR HEALTH MONITORING

(71) Applicants: LIFESIGNALS, INC., Milpitas, CA (US); BIOINTELLISENSE, INC., Golden, CO (US)

(72) Inventors: Saravanan BALASUBRAMANIAN, Bangalore (IN); Ajeeth MURALIDHARAN, Tamil Nadu (IN); Thomas DIETIKER, Las Vegas, NV (US)

(21) Appl. No.: 19/363,429

(22) Filed: Oct. 20, 2025

Related U.S. Application Data
(63) Continuation of application No. PCT/US24/33433, filed on Jun. 11, 2024.

(30) Foreign Application Priority Data
Nov. 10, 2023 (IN) 202311077035

(51) Int. Cl. A61B 5/1455; A61B 5/00

(52) U.S. Cl. CPC A61B 5/14552; A61B 5/6833; A61B 2562/12

(57) ABSTRACT

A patch for monitoring physiological data of a user is described herein and methods for using such patch. An exemplary patch comprises (a) a base configured to come in contact with a surface of a user; (b) one or more sensors operably coupled to the base, the one or more sensors configured to monitor the physiological data from the user; and (c) an electronic module in communication with the one or more sensors, the electronic module configured to receive the monitored physiological data. The base of such a patch may comprise one or more adhesive regions and one or more non-adhesive regions, and the one or more non-adhesive regions are configured to flatten or bulge between the adhesive regions. The sensors comprise SpO2 sensors, and each of the SpO2 sensors comprises a top structure, a bottom structure, and a substrate positioned therebetween.

### CROSS-REFERENCE

The present application is a continuation of International Patent Application No. PCT/US24/33433, filed Jun. 11, 2024, which claims the benefit of priority of Indian Patent Application No. 202311077035, filed Nov. 10, 2023, each of which is incorporated herein by reference in its entirety.

### BACKGROUND

Monitoring physiological conditions of patients has been an important component of health care. Although the monitoring can be performed periodically by health care professionals, the task is being managed by electronics that connect the patient to a computerized system for autonomous data storage, processing, presentation and retrieval. The autonomous monitoring of physiological condition has also become an important part of everyday life with the advent of the quantified-self movement.

Most sensors used in the monitoring physically contact the human body. While some sensors (e.g., a wrist monitor) can be used to track a person's physical activity or sleep patterns and collect associated data, cumbersome wires distributed throughout the body have traditionally been necessary to collect more meaningful physiological data.

### SUMMARY

Technologies disclosed herein may overcome drawbacks in the existing physiological monitoring systems. The technologies may miniaturize sensors/electrodes and data collection devices. Sensors and electrodes may be integrated on a printed circuit board. Data collection devices may be designed as portable or wearable devices. Wireless interfaces may be integrated on sensors/electrodes and data collection devices to facilitate physiological signal collection based on wireless communications technologies. Further, physiological signals may be sent from a data collection device to a server for signal analytics. Analytics results may be sent to a consumer or a patient or a healthcare provider.

In one aspect, disclosed herein is a patch for monitoring physiological data comprising (a) a base configured to come in contact with a surface of a user; (b) one or more sensors operably coupled to the base, the one or more sensors configured to monitor the physiological data from the user; and (c) an electronic module in communication with the one or more sensors, the electronic module configured to receive the monitored physiological data, where the base comprises one or more adhesive regions and one or more non-adhesive regions, and the one or more non-adhesive regions are configured to flatten or bulge between the adhesive regions.

In some embodiments, the sensors may comprise a plurality of clinical grade sensors. The sensors may comprise one or more of the following sensors: electrocardiogram (ECG), heart rate, heart rate variation, respiration, oxygen saturation (SpO2), temperature, accelerometer, gyroscope, hydration, and heart sound.

In some embodiments, the electronic module may comprise a wireless communication means. The wireless communication means may comprise one or more of the following: a near range communication means, a short range communication means, and a long range communication means. The wireless communication means may operate on one or more of the following protocols: a Bluetooth protocol, a Wi-Fi protocol, an ultra-wide band protocol.

In some embodiments, the patch may further comprise one or more island ring electrodes, wherein each of the island ring electrodes comprises an electrical contact configured to convey electrode data to the electronic module.

In some embodiments, the non-adhesive regions of the patch may comprise a stretchable material and a non-stretchable material. The patch may further comprise a top layer, a support adhesive layer, and a skin contact adhesive layer. The top layer, the support adhesive layer, and the skin contact adhesive layer may have offset against one another.

In some embodiments, the one or more sensors may comprise a SpO2 sensor, and the SpO2 sensor may comprise a top structure, a bottom structure, and a substrate positioned therebetween. The top structure may comprise a soft material, and the bottom structure may comprise a hard material. The soft material may be silicone. The hard material may be acrylonitrile butadiene styrene (ABS), polycarbonates (PC), or a combination thereof. The top structure and the bottom structure are injection-molded (i.e., formed by injection molding).

In some embodiments, the electronic module is encapsulated within a case. In some embodiments, the case comprises a floating rigid center connected to flexible extensions. In some cases, the flexible extensions house one or more island ring electrodes. In some embodiments, an airgap is located between the floating rigid center and the skin contact adhesive layer.

In some embodiments, the patch may further comprise one or more removable release liners configured to cover at least one part of the base. The removable release liners may be made of paper or plastic.

Another aspect disclosed herein is a method of monitoring physiological data of a user, the method comprising (a) contacting a surface of a user with a patch, wherein the patch comprises (1) a base configured to come in contact with a surface of a user; (2) one or more sensors operably coupled to the base, the one or more sensors configured to monitor the physiological data from the user; and (3) an electronic module in communication with the one or more sensors, the electronic module configured to receive the monitored physiological data, where the base comprises one or more adhesive regions and one or more non-adhesive regions, and the one or more non-adhesive regions are configured to flatten or bulge between the adhesive regions; and (b) monitoring physiological data of a user from the patch.

In some embodiments, the method further comprises (c) wirelessly transmitting the physiological data of the user to an external device.

In some embodiments of the method, the sensors comprise a plurality of clinical grade sensors. In some embodiments, the sensors comprise one or more of the following sensors: electrocardiogram (ECG), heart rate, heart rate variation, respiration, oxygen saturation (SpO2), temperature, accelerometer, gyroscope, hydration, and heart sound.

In some embodiments of the method provided herein, the electronic module comprises a wireless communication means. In some embodiments, the wireless communication means comprises one or more of the following: a near range communication means, a short range communication means, and a long range communication means. In some embodiments, the wireless communication means operates on one or more of the following protocols: a Bluetooth protocol, a Wi-Fi protocol, an ultra-wide band protocol.

In some embodiments, the patch further comprises one or more island ring electrodes, wherein each of the island ring electrodes comprises an electrical contact configured to convey electrode data to the electronic module.

In some embodiments, the non-adhesive regions comprise a stretchable material and a non-stretchable material. In some embodiments, the patch comprises a top layer, a support adhesive layer, and a skin contact adhesive layer. In some embodiments, the top layer, the support adhesive layer, and the skin contact adhesive layer are offset against one another.

In some embodiments, the sensors comprise SpO2 sensors, and each of the SpO2 sensor comprises a top structure, a bottom structure, and a substrate positioned therebetween. In some embodiments, the top structure comprises a soft material, and the bottom structure comprises a hard material. In some embodiments, the soft material is silicone. In some embodiments, the hard material is acrylonitrile butadiene styrene (ABS), polycarbonates (PC), or a combination thereof. In some embodiments, the top structure and the bottom structure are injection-molded.

In some embodiments, the patch further comprises one or more removable release liners configured to cover at least one part of the base. In some embodiments, the removable release liners are made of paper or plastic.

It shall be understood that different aspects of the present disclosure can be appreciated individually, collectively, or in combination with each other. Various aspects of the disclosure described herein may be applied to any of the particular applications set forth below. Other objects and features of the present disclosure will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference in their entirety.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a subject wearing a multi-parameter patch, in accordance with some embodiments;

FIG. 2 shows a non-limiting example of a multi-parameter wearable patch, in accordance with some embodiments;

FIG. 3 shows a non-limiting example of a multi-parameter wearable patch, in accordance with some embodiments;

FIG. 4 shows a non-limiting example of a multi-parameter wearable patch, in accordance with some embodiments;

FIG. 5 shows a non-limiting example of a multi-parameter wearable patch comprising one or more island ring electrodes, in accordance with some embodiments;

FIG. 6 shows a non-limiting example of a multi-parameter wearable patch comprising one or more island ring electrodes, in accordance with some embodiments;

FIG. 7 shows a non-limiting example of a multi-parameter wearable patch comprising an adhesive region and a non-adhesive region, in accordance with some embodiments;

FIGS. 8A and 8B show cross sectional views of a multi-parameter wearable patch, in accordance with some embodiments;

FIGS. 9A and 9B show cross sectional views of a multi-parameter wearable patch, in accordance with some embodiments;

FIG. 10 shows a cross sectional view of a multi-layer wearable patch, in accordance with some embodiments;

FIG. 11 shows a cross sectional view of a multi-layer wearable patch, in accordance with some embodiments;

FIG. 12 shows a non-limiting example of a multi-parameter wearable patch comprising an oxygen saturation (SpO2) sensor, in accordance with some embodiments;

FIG. 13 shows a non-limiting example of a SpO2 sensor, in accordance with some embodiments;

FIG. 14 shows a non-limiting example of a SpO2 sensor, in accordance with some embodiments;

FIGS. 15A, 15B, and 15C show visual indicators on a multi-parameter wearable patch, in accordance with some embodiments;

FIGS. 16A and 16B show non-limiting examples of a multi-parameter wearable patch, in accordance with some embodiments;

FIG. 17 shows a non-limiting example of a multi-parameter wearable patch placed on the chest of a subject, in accordance with some embodiments;

FIG. 18 shows a non-limiting example of a multi-parameter wearable patch placed on the chest of a subject, in accordance with some embodiments;

FIG. 19 shows a non-limiting example of a multi-parameter wearable patch placed on the chest of a subject, in accordance with some embodiments;

FIGS. 20A, 20B, and 20C show placement of a multi-parameter wearable patch comprising one or more release liners, in accordance with some embodiments;

FIGS. 21A and 21B show non-limiting examples of a multi-parameter wearable patch comprising paper release liners and plastic release liners, in accordance with some embodiments; and

FIGS. 22A and 22B show non-limiting examples of a multi-parameter wearable patch comprising plastic release liners, in accordance with some embodiments.

### DETAILED DESCRIPTION

Unless otherwise defined, all technical terms used herein have the same meaning as commonly understood by one of ordinary skill in the art to which this disclosure belongs. As used in herein, the singular forms “a,” “an,” and “the” include plural references unless the context clearly dictates otherwise. Any reference to “or” herein is intended to encompass “and/or” unless otherwise stated. As used herein, the term “about” in some cases refers to an amount that is approximately the stated amount. As used herein, the term “about” refers to an amount that is near the stated amount by 10%, 5%, or 1%, including increments therein. As used herein, the term “about” in reference to a percentage refers to an amount that is greater or less the stated percentage by 10%, 5%, or 1%, including increments therein. As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The technologies disclosed herein may miniaturize electrodes and data collection devices into a compact wireless platform (e.g., patch) that may be attached to a subject. The wearable patch may comprise a plurality of sensors and a communication interface which may wirelessly and directly communicate with an external device, such as a mobile device, capable of executing one or more applications. The plurality of sensors may be configured to collect different types of physiological data from the subject. The applications may be configured to activate, transfer data to, receive data from, and/or otherwise utilize data from different combinations of the plurality of sensors integrated in the patch. The patch may be activated by the external device to monitor different activities via the one or more applications, including cardiac care, sleep apnea, athlete fitness, infant monitoring, senior living, pet monitoring, gait analysis, mental stress, emotional states, and fall detection, among other activities described herein. The sensors may include clinical grade sensors. The patch may be configured to allow clinically-approved placement of the sensors on the subject. In some embodiments, the systems and methods disclosed herein may help simplify a health monitoring process and/or may help create an agile, portable health monitoring systems. In some embodiments, the systems and methods disclosed herein may enable widespread or ubiquitous health monitoring based on portable devices.

The external device may, based on the type of application it is executing, receive data from one or more sensors in the patch, and analyze such data to output a result. The result may be a progress, product, or other analytics of one or more monitored activities of the application. The result may be displayed by the external device, such as via an integrated display of the external device (e.g., mobile device display) or an external display coupled to the external device. Alternatively or in addition, the result may be output in a non-visual manner, such as via auditory signals (e.g., beeping), haptic signals (e.g., vibrations), olfactory signals (e.g., dispensing an odor) or other non-visual signals. In some embodiments, the result may be delivered to and outputted by a wearable device worn by the subject. For example, such wearable device may be a watch or a band. The results may be outputted in a visual manner, such as via a display of the wearable device, and/or in a non-visible manner, such as via a speaker or actuator of the wearable device. Any process described herein may occur in real-time.

The technologies disclosed herein may support various health monitoring procedures. Examples of the health monitoring procedures may include, but are not limited to: multi-day live monitoring where a subject may be monitored by a remote healthcare provider or a family member; in-patient monitoring where data is continuously displayed on patient monitors to facilitate patient monitoring; ambulatory out-patient monitoring where an ambulatory staff member may monitor physiological signals and correlate the physiological signals with symptoms; Holter monitoring where a subject's various electrical activity of cardiovascular system may last more than 24 hours; event monitoring where an event may be flagged either by a subject or others, e.g., by pressing a button and few minutes of data may be transmitted on occurrence of an event.

Beneficially, the patch provided herein may have sufficient flexibility to accommodate and enable the monitoring of different activities, even those uncontemplated at the time of manufacture of the hardware portion of the patch. Any user, such as individuals, entities, manufacturers, healthcare professionals, medical operators, and others, may design and engineer an application utilizing any combination of a plurality of sensors included in the patch to achieve an objective, including clinical objectives. Such applications may be created, prior to, simultaneously, or subsequent to manufacture of the hardware portion of the patch. The patch may comprise a processor and/or electronic module capable of controlling the plurality of sensors in the patch to comply with the requirements and instructions of the different applications executed by an external device in communication with the patch via a communication interface of the patch.

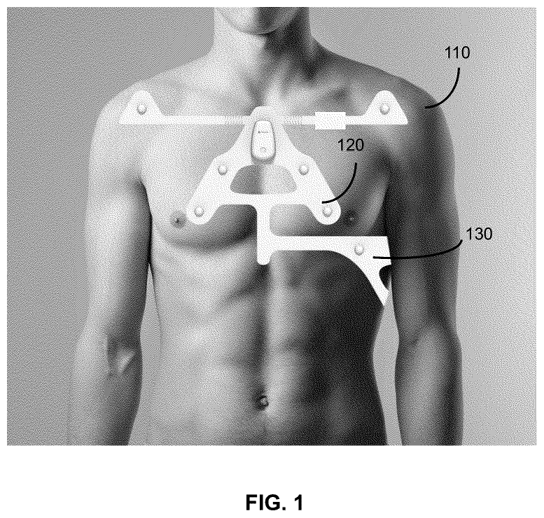

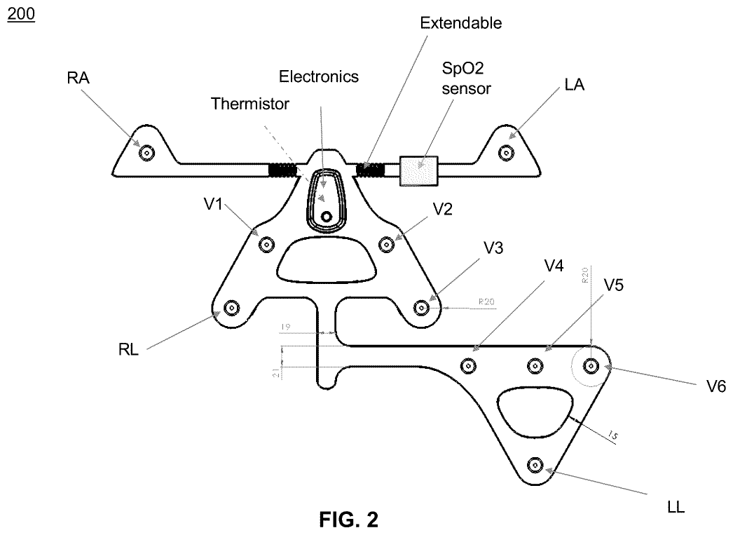

FIG. 1 shows a subject wearing a multi-parameter patch, in accordance with some embodiments. The patch 120 is positioned on the chest of the subject 110. The patch may be an integrated patch. The patch shown in FIG. 1 has 12-lead ECG capability and is shaped for Mason-Likar placement (see, e.g., V2-V6 at 130). By maintaining a shape that is compatible with 12-lead placement, the patch allows for more convenient set up on a subject. The position of the patch is to allow the sensor on the patch to obtain readings from the subject 110. For example, a patch might be located on the chest so that it is compatible with obtaining chest SpO2 readings. The size of the patch is also selected to be compatible with the desired use. For example, the patch in FIG. 1 is sized so as to comfortably fit on the chest of the subject. Patches may come in different sizes. For example, patches may be provided in a small, medium, and large size, which are selected to best fit a subject based on factors such as the subject's gender, BMI, or size generally, for example. Patches may also have extendable and retractable components, as described further in connection to FIG. 2, which further allow for tailoring the patch so that any body size can be accommodated. As a result, better accuracy in physiological readings is achieved relative to that which would result from using a patch without such features.

A patch may be designed for monitoring physiological data. In some embodiments, physiological data may include data related to cardiac functionality, respiratory functionality, pulmonary comorbidities, and positional data. Examples of physiological data include, but not limited to, heart rate, cardiac rhythms, respiration, blood saturation, body temperature, conductivity, impedance, resistance, motion, orientation, position, synaptic signals, neural signals, voice signals, vision or optical signals, electrocardiogramtriography, electroventriculography, intracardiac electrogram, electroencephalography, electrocorticography, electromyography, electrooculography, electroretinography, electronystagmography, electroolfactography, electroantennography, electrocochleography, electrogastrography, electrogastroenterography, electroglottography, electropalatography, electroarteriography, electroblepharography, electrodermography, electrohysterography, electroneuronography, electropneumography, electrospinography, and electrovomerography. Methods of using the patch can include, for example, a method of monitoring physiological data of a user, the method comprising (a) contacting a surface of a user with a patch, wherein the patch comprises (1) a base configured to come in contact with a surface of a user; (2) one or more sensors operably coupled to the base, the one or more sensors configured to monitor the physiological data from the user; and (3) an electronic module in communication with the one or more sensors, the electronic module configured to receive the monitored physiological data, where the base comprises one or more adhesive regions and one or more non-adhesive regions, and the one or more non-adhesive regions are configured to flatten or bulge between the adhesive regions; and (b) monitoring physiological data of a user from the patch.

A patch may comprise one or more sensors, the one or more sensors configured to monitor data (e.g., physiological, positional) from the user. For example, the number of electrodes may be 1, 2, 3, 4, 5, 6, 7, 8, 9, 10, 11, 12, 13, 14, 15, 16, 17, 18, 19, 20 or more than 20. The one or more sensors may each be a different type of sensor. Alternatively, the one or more sensors may comprise duplicate types of sensors (e.g., two temperature sensors). Sensors of the present disclosure can include, but is not limited to, electrocardiagraphy (ECG) (e.g., multi-lead ECG), tissue impedance, heart rate, heart rate variation, respiration (e.g., respiration rate and pattern), pulse oximetry (e.g., to detect peripheral capillary oxygen saturation (SpO2)), temperature, accelerometer (e.g., 3-axis), body position and motion, gyroscope, hydration, heart sound, cameras or other optical sensors, microphones or other auditory sensors, and the like.

The patch may comprise a base comprising a plurality of sensors configured to gather physiological data from the subject; a cover coupled to the base, where the cover may comprise one or more indicators associated with a preferred placement or orientation of the patch; an electronic module in communication with the plurality of sensors and the one or more indicators; and a signal transceiver in communication with the electronic module, where the signal transceiver is configured to wirelessly transmit and receive data. The base of a patch may comprise a flexible substrate. Alternatively, the base may comprise a substantially rigid substrate. Materials of the base may comprise a plastic material, an elastomeric material, and/or a silicone material. The base may be used for holding sensors and/or electronic components. In some embodiments, a surface of the base may be designed to contact with a user at a forehead, an arm, a chest, a leg, and a finger. Alternatively or in addition, a surface of the base may be designed to contact another layer, such as an adhesive layer for contacting the user. Alternatively or in addition, a surface of the base may be designed to contact or interface a cover layer, such as a protective cover of the base configured for protecting the base. Alternatively or in addition, a surface of the base may be designed to contact a body interface layer configured for coupling the base to the user. In some embodiments, a first surface of the base may comprise one or more electronics and a second surface of the base may be configured to interface a user or a layer configured to interface the user.

The base may be of any shape, size, or form. The patch may be in a small size. For example, the patch may comprise a maximum dimension equal to or smaller than about 5 centimeters (cm), 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. Alternatively, the patch may be greater than about 20 cm. The thickness of a patch may be equal or less than about 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, 4 cm, 5 cm, 6 cm, 7 cm, or 8 cm. Alternatively, the patch may have a thickness greater than about 8 cm. A patch may have a volume equal to or smaller than about 5 cm³, 10 cm³, 15 cm³, 20 cm³, 25 cm³, 30 cm³, 35 cm³, 40 cm³, 45 cm³, 50 cm³, 60 cm³, 70 cm³, 80 cm³, 90 cm³, or 100 cm³. Alternatively, the patch may have a volume greater than about 100 cm³.

The mass of the patch may be equal or less than about 1 gram, 2 grams, 3 grams, 4 grams, 5 grams, 6 grams, 7 grams, 8 grams, 9 grams, 10 grams, 11 grams, 12 grams, 13 gram, 14grams, 15 grams, 16 grams, 17 grams, 18 grams, 19 grams, 20 grams, 21 gram, 22 grams, 23 grams, 24 grams, 25 grams, 26 grams, 27 grams, 28 grams, 29 grams, 30 grams, 31 gram, 32 grams, 33 grams, 34 grams, 35 grams, 36 grams, 37 grams, 38 grams, 39 grams, 40 grams, 41 gram, 42 grams, 43 grams, 44 grams, 45 grams, 46 grams, 47 grams, 48 grams, 49 grams, 50 grams, 60 grams, 70 grams, 80 grams, 90 grams, 100 grams, 110 grams, 120 grams, 130 grams, 140 grams, 150 grams, 160 grams, 170 grams, 180 grams, 190 grams, or 200 grams. Alternatively, the patch may have a mass greater than about 200 grams. A thin, light patch may provide various benefits. One benefit may be to allow a user to easily carry the patch. Another exemplary benefit may be to allow a user to use the patch on a daily basis. Another benefit is low energy consumption. Another benefit may be to allow easy configuration, or handling of the patch. Another benefit may be to provide a patch without need to worry about wiring as the patch may comprises a wireless communication capability.

The patch is generally wearable. In some cases the patch may be held onto the skin with an adhesive. In some embodiments, where an electrical signal is being measured, e.g., for ECG or EEG, some or all of the adhesive may be electrically conducting, for example comprising silver and or silver chloride. In other cases, the adhesive may be electrically non-conductive. The patch adhesive may be either wet or dry. In some embodiments, the patch may be held in place with straps or clips or may be incorporated into a piece of wearable clothing such as a hat, gloves, socks, shirt, or pants. In some embodiments, the patch may be implanted. The patch may be placed on any suitable part of the body depending on the physiological signal and the condition to be measured. For ECG monitoring, for example, a single patch may be used, the patch may be placed on the upper-chest area. For EEG monitoring, e.g., for monitoring sleep apnea, multiple patches may be placed on the head.

A patch may comprise an electronic module in communication with the one or more electrodes and one or more sensors. The electronic module may be configured to receive the monitored data from the electrodes and sensors. The electronic module may be operatively coupled to a wireless transmission mechanism, such as an antenna.

The patch and components (e.g., electrodes, sensors) therein may be configured such that an orientation and placement of patch generates meaningful or high quality data. High quality data as used herein may refer to precise, accurate, interpretable, and/or reproducible data. A single patch of the present disclosure may be sufficient to collect high quality data. A placement of the patch on the human body may be of importance for obtaining a high quality data. For example, high quality data may be obtained when the patch is placed near the upper left chest or at a center of the chest. A patch placed near a center of the chest may be centered over the sternum. A patch placed near a center of the chest may be centered over the sternum and no lower than the xiphoid process.

An orientation of the patch may be of importance for obtaining high quality data. For example, when the patch is placed near the upper left chest, electrodes arranged in a normal orientation (e.g., relative to a longitudinal axis of the user) may enable acquisition of high quality data. A normal orientation as used herein may refer to an arrangement of electrodes where electrodes are arranged in a substantially rectangular shape relative to a longitudinal axis of the user. Electrodes arranged in a normal orientation may have a virtual line going through a left arm (i.e., LA) electrode and a right arm (i.e., RA) electrode, which is substantially perpendicular to a longitudinal axis of the user. Electrodes arranged in a normal orientation may have a virtual line going through a left leg (i.e., LL) electrode and a right leg (i.e., RL) electrode, which is substantially perpendicular to a longitudinal axis of the user. Electrodes arranged in a normal orientation may have a virtual line going through a LA and a LL electrode, which is substantially parallel to a longitudinal axis of the user. Electrodes arranged in a normal orientation may have a virtual line going through a RA and a RL electrode, which is substantially parallel to a longitudinal axis of the user. Such configuration of electrodes enables acquisition of high quality data when the patch is placed near the upper left chest. Such configuration of electrodes may enable acquisition of high quality data when the patch is placed near a center of the chest.

In some embodiments, a different orientation of the patch may be preferred relative to a longitudinal axis of the user. For example, electrodes arranged in a tilted orientation (e.g., relative to a longitudinal axis of the user) may enable acquisition of high quality data. A tilted orientation as used herein may refer to an arrangement of electrodes that is rotated about 15°, 30°, 45°, 60°, 75°, 90°, 120°, 150°, 180° or more clockwise from the normal orientation. A tilted orientation as used herein may refer to an arrangement of electrodes that is rotated about 15°, 30°, 45°, 60°, 75°, 90°, 120°, 150°, 180° or less clockwise from the normal orientation. A tilted orientation as used herein may refer to an arrangement of electrodes that is rotated about 15°, 30°, 45°, 60°, 75°, 90°, 120°, 150°, 180° or more counterclockwise from the normal orientation. A tilted orientation as used herein may refer to an arrangement of electrodes that is rotated about 15°, 30°, 45°, 60°, 75°, 90°, 120°, 150°, 180° or less counterclockwise from the normal orientation. A tilted orientation as used herein may refer to an arrangement of electrodes that is rotated 15°, 30°, 45°, 60°, 75°, 90°, 120°, 150°, or 180° clockwise or counterclockwise from the normal orientation. In some embodiments, a tilted orientation of electrodes may be arranged in a diamond shape relative to a longitudinal axis of the user. Electrodes arranged in a tilted orientation may have a virtual line going through a LA electrode and a RL electrode be substantially parallel to a longitudinal axis of the user. Electrodes arranged in a tilted orientation may have a virtual line going through a LL and RA electrode be substantially perpendicular to a longitudinal axis of the user. Electrodes arranged in a tilted orientation may have a virtual line going through a LL electrode and a RA electrode be substantially parallel to a longitudinal axis of the user. Electrodes arranged in a tilted orientation may have a virtual line going through a LA and RL electrode be substantially perpendicular to a longitudinal axis of the user.

Patches, and/or associated software thereof, of the present disclosure may be configured to generate, and/or be capable of generating, clinical grade data or medical grade data. For example, data generated by the patches may meet the requirements of one or more accepted clinical or medical standards, models, formats, terminologies, and/or guidelines. In some embodiments, the standards may include different types of standards, such as measurement standards (e.g., molecular biomarkers, patient-reported outcomes, observer-reported outcomes, clinician-reported outcomes), methods standards (e.g., disease models, in vitro models, clinical trial simulation tools), and data standards (e.g., clinical data standards for certain therapeutic areas). Such standards may be created, accredited, or supported by one or more entities or consortiums, such as the Clinical Data Interchange Standards Consortiums (CDISC), Critical Path Institute (C-Path), Coalition for Accelerating Standards and therapies (CFAST), and Food and Drug Administration (FDA).

Different requirements may be associated with different therapeutic areas (e.g., cardiovascular, asthma, diabetes, pain) to qualify as clinical grade data. The patch, and/or associated software thereof, may be configured to generate clinical grade data in one or more different therapeutic areas. In some embodiments, the patch, and/or associated software thereof, may satisfy requirements presented by one or more open standards (e.g., CDISC Study Data Tabulation Model (SDTM) standards, Fast Healthcare Interoperability Resources (FHIR) standards, Institute of Electrical and Electronics Engineers (IEEE) standards) for clinical grade data. Alternatively or in addition, the patch, and/or associated software thereof, may satisfy requirements presented by one or more guidelines (e.g., Continua Design Guidelines). Alternatively or in addition, the patch, and/or associated software thereof, may satisfy requirements presented by one or more regulatory entities. In some embodiments, the patches, and/or associated software thereof, may be configured to acquire, and/or be capable of acquiring, data using an acquisition method or procedure (e.g., duration, placement of sensors, orientation of sensors, types of sensors) that satisfies one or more accepted clinical or medical standards or guidelines.

The electronic module may comprise a multi-chip module or application-specific integrated circuit (ASIC) to integrate most of the needed functions into a single module. The ASIC may be a single chip device. In some embodiments, additional components may be added to the ASIC as needed. The electronic module may comprise, or otherwise be electrically coupled to, one or more of the following: sensor interface, processing unit, and wireless communication interface. Optionally, the electronic module may comprise one or more optional units. For example, the optional units may include, but is not limited to the following: instrumentation amplifiers for ECG and/or respiration signals, signal generation units for impedance variation measurement, right leg driver circuits, antennae, transimpedance amplifiers with LED driver circuits for SpO2, micro-processors, FLASH memory, LED indicators, voltage and temperature sensors, power management units, and defibrillation (or voltage surge) protection circuits with resistors and clamping diodes. The electronic module may comprise other electronic components, such as resistors, capacitors, transcoders, and connectors, to facilitate electrical connection. Alternatively or in addition, the aforementioned components may be located elsewhere on the patch and the electronic module may be in communication with the additional components.

The electronic module may comprise a wireless communication interface. The wireless communication interface may comprise one or more of the following: a near range communication mechanism, a short range communication mechanism, and/or a long range communication mechanism. A wireless communication unit may operate on one or more of the following protocols: a Bluetooth protocol, a Wi-Fi protocol, an ultra-wide band (UWB) protocol, and a narrowband protocol. The wireless communication interface may comprise one or more radio transmitters, receivers, and/or transceivers. The radio may enable the patch to communicate with other devices through a wireless link. The radio may be configured to wirelessly transmit and/or receive data. The radio may be configured to wirelessly transmit data to external devices and/or receive data from the external devices. The data collected by the patch may be unaltered or minimally processed physiological signals. When the data is transmitted to external devices or other devices

The radio may be a single-mode, dual-mode, triple-mode, or quad-mode radio. The radio may utilize any known bandwidth, e.g., narrowband, wideband, ultra-wideband, broadband. The radio may communicate through Wi-Fi, Bluetooth, wireless sub, and the like. In some embodiments, the radio may be a triple-mode hybrid radio. The triple-mode hybrid radio may utilize Wi-Fi, Medical band, and/or ultra-wideband bandwidths. The triple-mode hybrid radio may seamlessly transition between the three modes to maintain link integrity. The triple-mode hybrid radio may select a lowest power option when more than one option is available. Alternatively, the radio may be a single-mode or dual-mode radio. For example, the single-mode radio may utilize Wi-Fi. For example, the single mode radio may utilize Medical band bandwidths. For example, the single-mode radio may utilize ultra-wideband bandwidths. In some embodiments, the wireless communication interface may comprise at least one narrowband radio transmitter, receiver, and/or transceiver, and at least one ultrawideband radio transmitter, receiver, and/or transceiver. The wireless communication interface may be operably coupled to one or more antennae, such as wireless transmission mechanism.

The electronic module may comprise a processing unit. In some embodiments, the processing unit may be communicatively coupled to the sensor interface and the wireless communication interface. The processing unit may be further communicatively coupled to other electrical components, such as power storage medium (e.g., to receive, distribute, and/or manage power) and data storage medium (e.g., to coordinate and store application instructions and data, to coordinate and store sensor data, to coordinate and store programmable instructions of one or more processors). The power storage medium and the data storage medium may be integrated in the electronic module. Alternatively, the power storage medium and the data storage medium may be external to the electronic module. The processing unit may be further communicatively coupled to all or parts of the one or more optional units, such as a power management unit.

The processing unit may comprise one or more processors and a memory operably coupled to the one or more processors to execute systems and methods of the present disclosure. The processing unit may be configured to communicate with the one or more sensors via the sensor interface, such as to receive sensor data from and/or transmit instructions to the sensors (or sensor components). The processing unit may be configured to communicate with one or more external devices via the wireless communication interface, such as to receive application instructions from and/or transmit sensor data to the one or more external devices.

The processing unit may coordinate communication between an application being executed (or implemented) on an external device and a selected combination of one or more sensors. Different sensors in the plurality of sensors may be configured to collect different types of data from the subject. Different applications executed on the one or more external devices may be configured to activate, transfer data to, receive data from, and/or otherwise utilize data from different combinations of sensors integrated in the patch. The patch, and/or a selected combination of sensors therein, may be activated by an external device via one or more different applications. Different applications may utilize the different combinations of sensors to achieve one or more application-specific objectives. Such objectives may include, for example, the monitoring, tracking, and/or detection of one or more activities.

For example, the objectives may include, but are not limited to, cardiac care, monitoring and detecting cardiac electrical activity, monitoring and detecting cardiac rhythm assessment, monitoring and detecting mechanical heart conditions, monitoring and detecting respiration, monitoring and detecting lead-off states, monitoring skin temperature, monitoring blood oxygen levels, monitoring and detecting sleep apnea, monitoring and detecting snoring, monitoring and detecting sleep positions, monitoring and detecting sleep patterns, monitoring and detecting sleep REM activity, monitoring athlete fitness state, monitoring and detecting fitness activities (e.g., running, walking, rowing, planking), monitoring and detecting activity intensities and patterns, infant monitoring, pet monitoring, senior living, monitoring gait, monitoring and detecting mental stress, monitoring and detecting emotional states, monitoring and detecting falls, monitoring and detecting positional state, and monitoring and detecting other motion, among other objectives described herein.

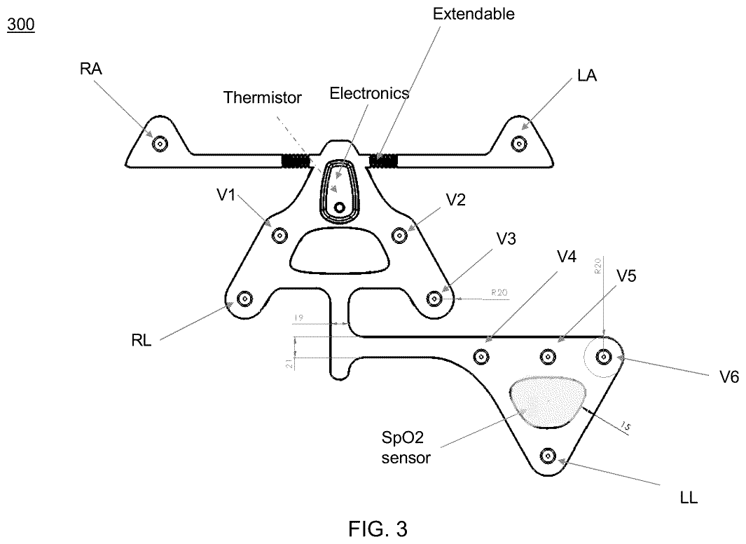

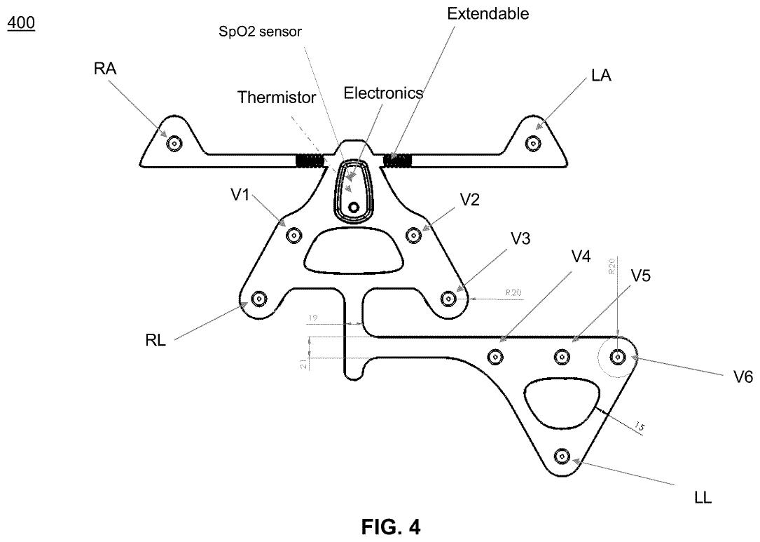

FIG. 2 shows a non-limiting example of a multi-parameter wearable patch, in accordance with some embodiments. The patch 200 may comprise a plurality of sensors for monitoring physiological parameters of the subject, including but are not limited to electrocardiogram (ECG), skin temperature, body temperature, heart rate, respiration rate, SpO2, pulse rate, non-invasive blood pressure (NIBP), body posture, and body activity. As illustrated, the patch 200 comprises a plurality of electrodes for ECG monitoring. In some embodiments, the patch may comprise a one-lead, a two-lead, a three-lead, a four-lead, a five-lead, a six-lead, a seven-lead, an eight-lead, a nine-lead, a ten-lead, an eleven-lead, or a twelve-lead ECG monitoring setup. In some embodiments, the patch may comprise a three-lead, a five-lead, a six-lead, a twelve-lead ECG monitoring setup. As illustrated, the patch comprises a twelve-lead ECG monitoring setup that includes ten electrodes on the patch. Four electrodes on the patch correspond to right arm (RA), left arm (LA), right leg (RL), and left leg (LL), and are configured to gather information sufficient to generate limb leads for some specific application. The patch also includes one or more expandable parts, for example, between the ECG electrodes LA and RA. The expandable parts provide flexibility such that the patch may be used to different sized subject bodies. Six electrodes on the patch, namely V1, V2, V3, V4, V5, and V6 correspond to precordial leads. The patch also comprises a SpO2 sensor located between the two ECG electrodes LA and RA. The electronic module, including a thermistor, is located at the center of the patch. In FIG. 2, the sensor is located on the left intercoastal side, between the RA and LA electrodes, but is offset so that it is closer to LA. Sensors may also be located, for example, further to the left or right of the location in FIG. 2. For example, in one embodiment, the sensor is still between the RA and LA electrodes, but offset closer to the RA electrode than the LA electrode. Sensors may also be located at other locations, such as in FIG. 3 or FIG. 4.

FIG. 3 shows a non-limiting example of a multi-parameter wearable patch, in accordance with some embodiments. The patch comprises a twelve-lead ECG monitoring setup and a SpO2 sensor. Different from the patch design as illustrated in FIG. 2 where the SpO2 sensor is located between the two ECG electrodes LA and RA, here, the SpO2 sensor is surrounded by four ECG electrodes LL, V4, V5, and V6. It should be noted that SpO2 sensors in FIG. 2 and FIG. 3 are for illustrative purposes without limiting the scope of the disclosure. Any other sensors may be located at the position of SpO2 sensor as illustrated in FIG. 2 and FIG. 3.

FIG. 4 shows a non-limiting example of a multi-parameter wearable patch, in accordance with some embodiments. The patch comprises a twelve-lead ECG monitoring setup and a SpO2 sensor. Different from the patch design as illustrated in FIG. 2 where the SpO2 sensor is located between the two ECG electrodes LA and RA, here, the SpO2 sensor is positioned under the electronic module of the patch and positioned at the sternum of the subject. It should be noted that the positions of SpO2 sensors in FIGS. 2-4 are for illustrative purposes without limiting the scope of the disclosure. The wearable patch may comprise two or more SpO2 sensors that are located at the positions as illustrated in FIGS. 2-4 or other locations on the patch. For example, the patch can be shifted left or right, relative to what is shown, or it may be raised higher or lower relative to what is shown in the exemplary embodiments provided in FIGS. 2-4. Any other sensors may be located at the position of SpO2 sensor as illustrated in FIGS. 2-4.

The wearable patch may be applied to subjects who need critical care as well as non-critical care. The patch design (e.g., expandable parts and flexible patch materials) may allow it to fit subjects with different sizes and different ages. In some embodiments, the patch may be used on pediatric, adult subjects and adolescent subjects. The wearable patch may be used in a variety of health care settings including clinical facilities, emergency or non-emergency transport vehicles, and homes. When subjects are in clinical facilities, the patch may allow in-hospital real-time monitoring. Subjects wearing the patch may receive the same level of monitoring as using health monitoring systems. Yet, subjects may enjoy significant flexibility of regular activities without being confined in patient bed and connected to the bulky monitoring systems. When subjects are at home, the patch may transmit physiological data to healthcare providers and allow them to monitor remote subjects real-time or near real-time.

The patch may be disposable. In some embodiments, the patch may be of single use. In some embodiments, the patch may be of multiple use. In some embodiments, the patch may comprise some reusable materials and some disposable materials. In some embodiments, the patch may be reusable. The subject may apply the patch to the body and when the monitoring is complete, remove the patch. In some embodiments, the patch may be used for 4 hours, 8 hours, 12 hours, 16 hours, 20 hours, or 24 hours. In other embodiments, the patch may be used for 1 day, 2 days, 3 days, 4 days, 5 days, 6 days, 7 days, 8 days, 9 days, 10 days. 11 days, 12 days, 13 days, 14 days or 15 days. Alternatively, the patch may be used for more than 15 days.

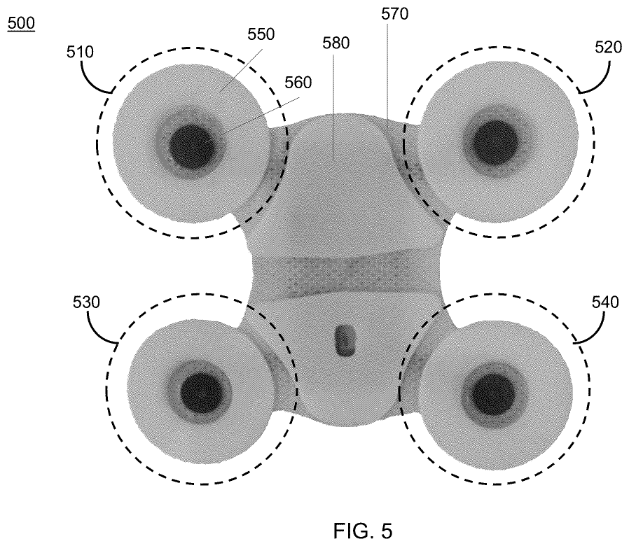

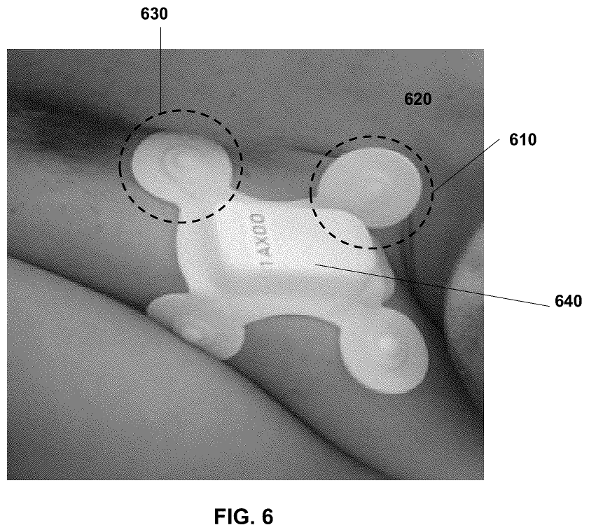

The patch may be an integrated patch. The wires and leads may be integrated into the patch, providing an advantage over those which traditionally require cumbersome wires in order to collect meaningful data. In some embodiments, the patch may have different designs to fit subjects with different genders, body sizes, and body shapes. For example, the patch may have different sizes (e.g., small, medium, and large) that fit subjects with different body sizes. In addition or alternatively, the patch may comprise or be made of expandable materials such that it may be easily stretched and applied to the subject body. In some preferred embodiments, the patch may comprise one or more of “island ring electrodes” for improved flexibility and reduced motion artifacts. The island ring electrode design will be explained in detail below in accordance with FIGS. 5 and 6. The sensors and electrodes within the patch may also have different sizes to fit subjects with different genders, body sizes (e.g., body mass index), and inner-wear sizes. The patch may have an intuitive design that enables sensors and electrodes to get into proper body locations automatically. In some embodiments, the patch design may be symmetrical. For example, ECG electrodes V1, V2, V3 and RL may be located at the center portion of the patch (see FIG. 2, FIG. 3, and FIG. 4). The symmetrical design may be easier for subject to align the electrodes and wear the patch. In some other embodiments, ECG electrodes V3 and V4 may not be connected. Instead, electrode V4 may be physically connected to the middle of electrodes RL and V3, to accommodate different breast sizes of female subjects.

As discussed above, the multi-parameter wearable patches may comprise one or more electrodes. Electrodes can be zinc, copper, lead, or silver with or without conductive gel. For example, the patch can comprise one or more silver/silver chloride electrodes (e.g., Ag/AgCl) with conductive hydrogel. The electrodes can have any shape or configuration. For example, electrodes may be small in shape, such as less than an inch in size (e.g., ¼ inch diameter, ⅓ inch diameter, ½ inch diameter, etc.). The electrodes may be ring-shaped electrodes or they may be disc-shaped electrodes, such as in FIG. 5 (e.g., 560).

In a preferable embodiment, the electrodes are configured and positioned as “island ring electrodes,” a term used herein to refer to the configuration and positions of the electrodes as an extension of the patch, which is exemplified by FIG. 5. FIGS. 5 and 6 show non-limiting examples of a multi-parameter wearable patch comprising one or more island ring electrodes, in accordance with some embodiments. FIG. 5 provides a view of a patch from the bottom side, which makes contact with skin on the subject. As shown in FIG. 5, island ring electrodes (e.g., 510) comprise small electrodes 560 surrounded by a ring-shaped portion 550. The ring-shaped portion 550 may comprise a flexible material, such as an elastic or mesh material. It may also contain an adhesive material or layer, such as in FIG. 5, where the adhesive layer is located at 550 and at 580.

As illustrated in FIG. 5, for example, the wearable patch 500 comprises four island ring electrodes 510, 520, 530, and 540, each of which form an extension of the patch, and which may be suited for patch form factor. The patch in FIG. 5 and the patch in FIG. 6 both have four island ring electrodes evenly dispersed and projecting outward from the center of the patch.

However, it should be understood that this is only an exemplary embodiment of the island electrode design. The patch can have any number of electrodes, which can be configured as island ring electrodes which extend in any direction. The island ring electrodes may provide optimal flexibility when the patch is applied to body contours, reduce shear stress and motion artifacts (see, e.g., FIG. 6). Hence, the lifetime of the patch as well as the quality and reliability of collected physiological signals may be increased. In FIG. 6, for example, the island ring electrodes (e.g., 630, 610) can be seen in contact with the chest of the subject 620. One of the island ring electrodes 610 is bent, but the patch is still securely attached to the subject 620.

As shown in FIG. 6, the patch may be sized such that it fits comfortable on one side of the chest of the subject 620. FIG. 6 provides a view of the patch from the top, which is where the external, rigid case of the patch is located, as seen on the chest of a subject 620. The central, rigid portion of the patch (e.g., 640) is placed on the left side of the subject's chest, which is proximate to the heart and therefore a position which is helpful in obtaining physiological readings.

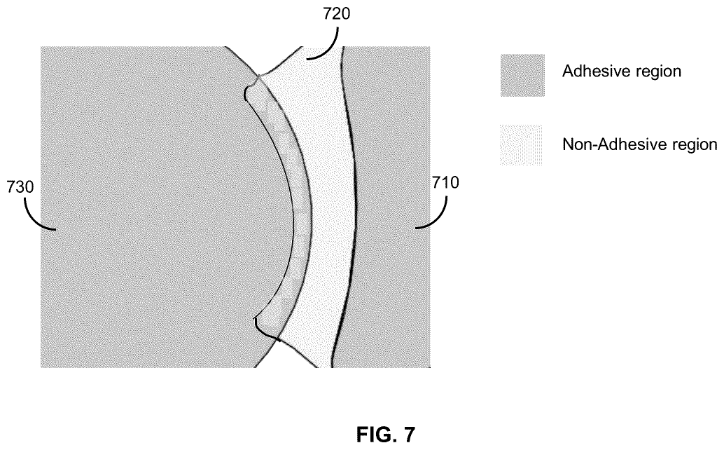

The island ring electrode design comprises one or more electrode areas and one or more non-electrode areas that are separated from the electrode areas. In some embodiments, the electrode areas and non-electrode areas may be adhesive regions (e.g., 710 and 730) and separated from each other by non-adhesive regions (e.g., 720). The non-adhesive region may comprise, for example, a soft, mesh material, a foam material, a non-woven fabric, or a flexible polymer film (e.g., a thin flexible polymer film). The electrodes may be ECG electrodes and/or other sensors (e.g., SpO2 sensor). In some embodiments, other sensors may be located at adhesive regions, and separated from each other by non-adhesive regions. FIG. 7 shows a non-limiting example of a multi-parameter wearable patch comprising an adhesive region and a non-adhesive region, in accordance with some embodiments. Such regions are visible in the island ring electrode design of the patch in FIG. 5. In FIG. 5, an adhesive region is at 550, and a non-adhesive is at 570. The non-adhesive region might be a cloth or mesh material, as provided in FIG. 5. Non-limiting examples of material for the adhesive layer include synthetic rubber, acrylate, silicone, or any other suitable material. In some embodiments, the adhesive portion comprises a backing material with an adhesive layer. For example, medical tape or a similar adhesive suitable for skin contact may be used in adhesive region of the patches. A nonlimiting example on the market is 3M 4077 Medical Tape, which has an elastic backing with a tackified acrylic adhesive.

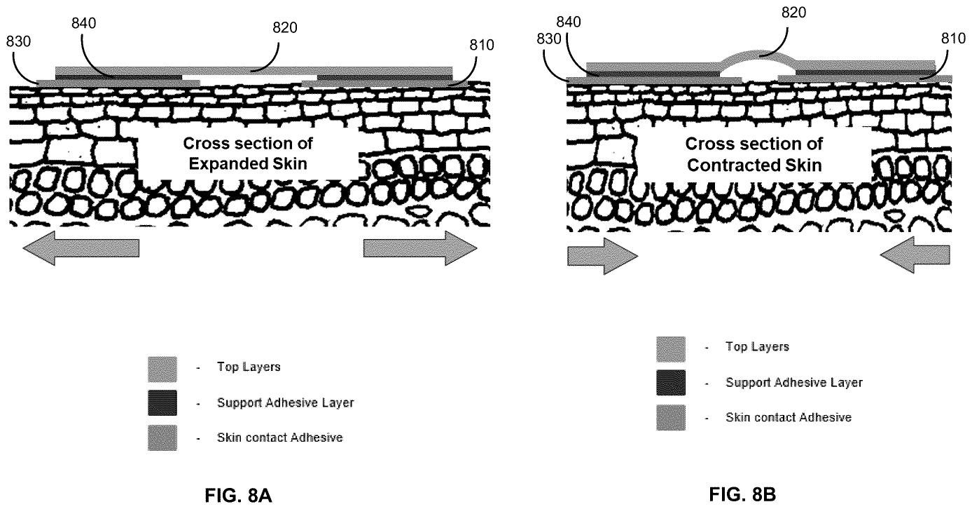

FIGS. 8A and 8B show cross sectional views of a multi-parameter wearable patch, in accordance with some embodiments. FIG. 8A shows a cross section of expanded skin to which a patch with a ring island electrode is applied. FIG. 8B shows a cross section of contracted skin to which a patch with a ring island electrode is applied. When the patch is applied to the subject body, the island ring electrode with adhesive regions and non-adhesive regions may support expansion and contraction of skin without affecting the adhesion. When the skin is expanded or stretched as illustrated in FIG. 8A, the non-adhesion region 820 is flattened, and two adhesion regions 810 and 830 are attached to the skin. When the skin is contracted, the non-adhesion region bulges between the two adhesion regions. The attachment of the adhesion regions to the skin is not affected.

The non-adhesive region in the island ring electrode may comprise both a stretchable layer and a non-stretchable layer between the adhesive regions. The stretchable layer 570 may be made of a flexible or stretchable material, for example, nonwoven fabric. In FIG. 6, the non-stretchable layer 640 is visible. In FIG. 5, the stretchable layer is visible (e.g., 570). In embodiments of the patch provided herein, the stretchable material/layer and the non-stretchable material/layer may have varying flexibilities. The stretchable layer is more flexible than the non-stretchable layer. In some embodiments, the stretchable layer is about: 5%, 10%, 15%, 20%, 25%, 30%, 35%, 40%, 45%, 50%, 55%, 60%, 65%, 70%, 75%, 80%, 85%, 90%, or 95% more flexible than the non-stretchable layer.

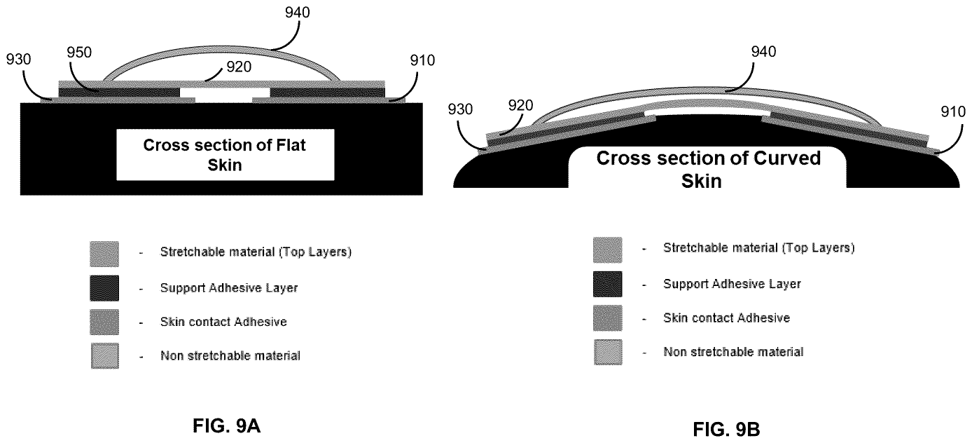

FIGS. 9A and 9B show cross sectional views of a multi-parameter wearable patch, in accordance with some embodiments. FIG. 9A shows a cross section of expanded skin to which a patch with a ring island electrode is applied. FIG. 9B shows a cross section of contracted skin where a patch with a ring island electrode is applied. Layers with stretchable material and non-stretchable material, and a skin contact adhesive layer are visible in FIG. 9A and FIG. 9B. In some embodiments, the stretchable layer is more flexible than the non-stretchable layer. In some embodiments, the stretchable layer is about 5%, 10%, 15%, 20%, 25%, 30%, 35%, 40%, 45%, 50%, 55%, 60%, 65%, 70%, 75%, 80%, 85%, 90%, or about 95% more flexible than the non-stretchable layer. As shown in FIG. 9A, for example, the non-adhesion region comprises both a stretchable layer 920 and a non-stretchable layer 940. When the skin is stretched, the stretchable layer 920 is flattened between two adhesion regions 910 and 930. The non-stretchable layer 940 maintains the structure of the patch and is less distorted than the stretchable material during movement. When the skin is contracted as illustrated in FIG. 9B, the stretchable layer 920 bulges between the two adhesion regions 910 and 930. The attachment of the adhesion regions to the skin is not affected. As a result, a subject may be in motion and the position and adherence of the patch will not be jeopardized, which is advantageous over other patches which do not contain the configuration and layers provided herein.

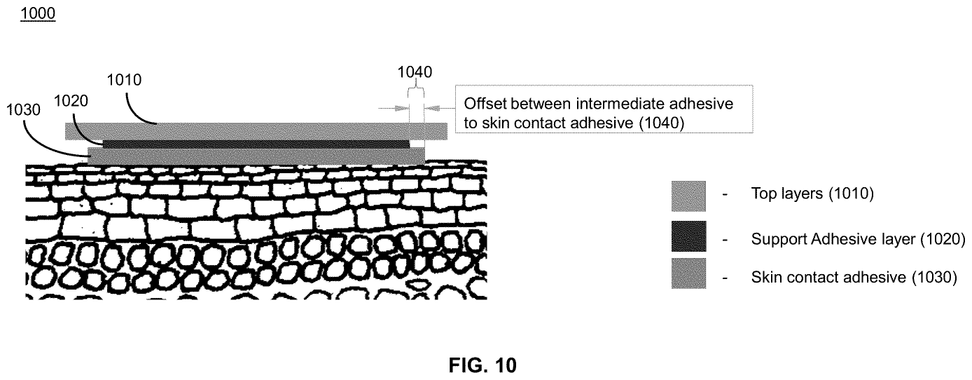

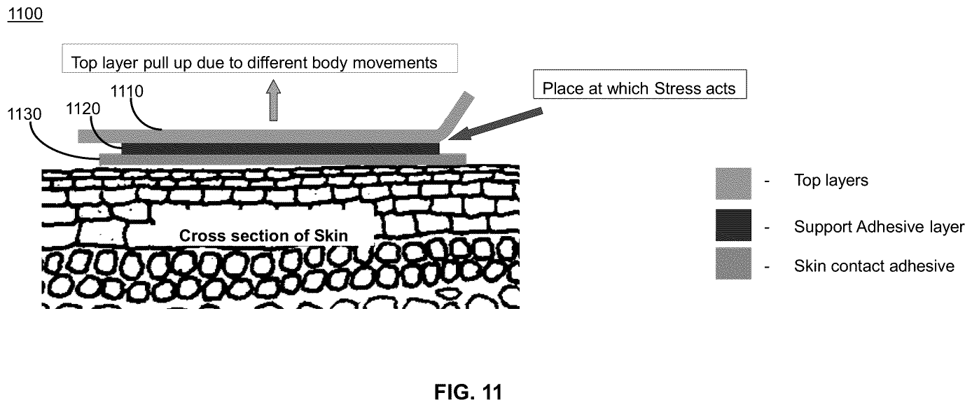

FIG. 10 shows a cross sectional view of a multi-layer wearable patch, in accordance with some embodiments. As illustrated, the patch 1000 has three layers: a top layer 1010, a support adhesive layer 1020, and a skin contact adhesive layer 1030. The support adhesive layer 1020 may sandwich the skin contact adhesive layer 1030 and the top layer 1010. The three layers 1010, 1020, and 1030 may have different sizes and/or offsets between one another. For example, the support adhesive layer 1020 may have an offset against the skin contact adhesive layer 1030 and the top layer 1010. As shown in FIG. 10 and FIG. 11, the support adhesive layer can be smaller than the top layer and the skin contact adhesive layer. Such an offset 1040 is important because any stress on the top layer (e.g., during body motion) will not be transferred to the edges of the skin contact adhesive, which can reduce the tendency of the skin contact adhesive layer to peel off at its edges. This is further illustrated in FIG. 11.

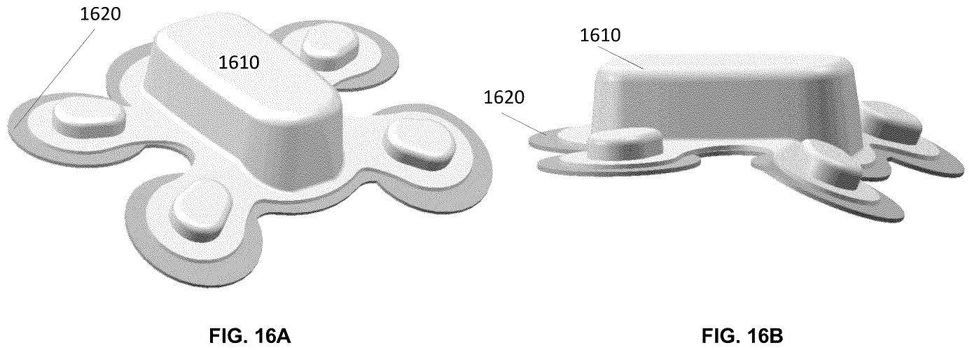

FIG. 11 similarly shows a cross sectional view of a multi-layer wearable patch, in accordance with some embodiments. As illustrated, the patch 1100 has three layers, a top layer 1110, a support adhesive layer 1120, and a skin contact adhesive layer 1130. The top layer 1110 comprises stretchable material but may further contain a non stretchable layer, which is not visible. This non-stretchable material is shown in FIG. 9A at 940 and can be seen in FIG. 6 at 640 and FIG. 16A and FIG. 16B at 1610. The support adhesive layer 1120 may sandwich the skin contact adhesive layer 1130 and the top layer 1110. The support adhesive layer 1120 may have an offset against the skin contact adhesive layer 1130 and the top layer 1110. As discussed above and further demonstrated by FIG. 11, the edges of skin contact adhesive layers are commonly prone for peeling off. The offset design may eliminate this problem. Any stress applied to the top layer caused by body motion may not be transferred to the edges of the skin contact adhesive layer. Non-limiting examples of material for the skin contact adhesive layer 1130 include synthetic rubber, acrylate, silicone, or any other suitable material. Non-limiting examples of material for the support adhesive layer 1120 include non-woven material, thin flexible polymer films or a foam material. Non-limiting examples of material for the stretchable top layer 1110 include non-woven material, thin flexible polymer film or a foam material. Non-limiting materials for the non stretchable material at the top layer include rigid polymer sheets. The non stretchable material at the top layer may further include electronic components such as printed circuit board, battery, conductive wiring, or metal films.

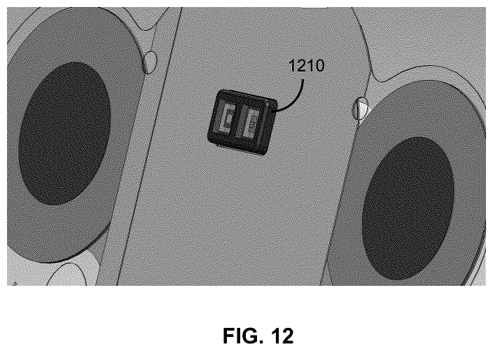

FIG. 12 shows a non-limiting example of a multi-parameter wearable patch comprising an oxygen saturation (SpO2) sensor, in accordance with some embodiments. The wearable patch may comprise one or more optical sensors. As illustrated in FIG. 12, the optical sensor 1210 may be a chest-based or abdomen based SpO2 sensor. The optical sensor may have a projected design that provides a positive pressure on the patient body, which is required for maintaining contact between the sensor and the body. Such design may minimize the impact to the sensor performance caused by the body motion and different body contours. The projected sensor may be made of a soft and flexible material that may be in direct contact with the patient body, allowing the patient to wear the patch for a long time. The soft and semi-flexible material may provide a positive pressure on the body and avoid optical crosstalk when the subject wears the patch.

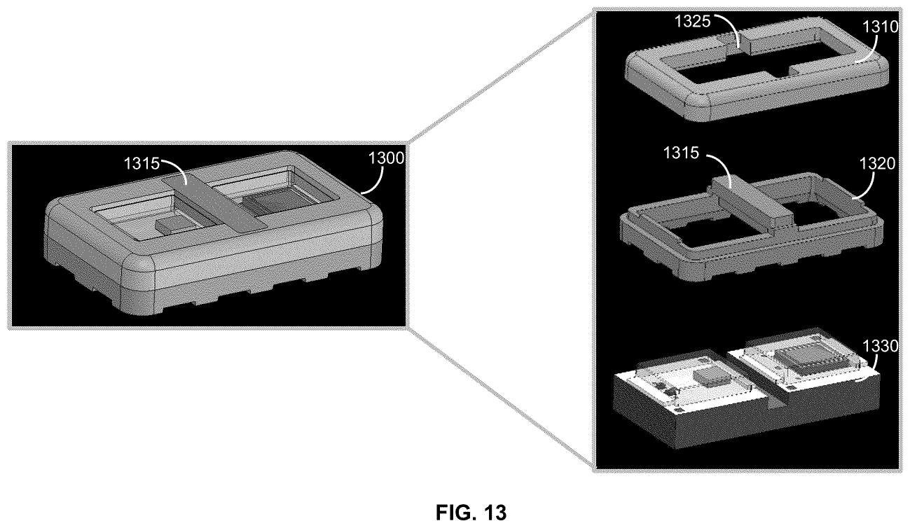

FIG. 13 shows a non-limiting example of a SpO2 sensor, in accordance with some embodiments. As illustrated, the SpO2 sensor 1300 comprises three materials. As shown, the sensor can be further broken into three general components: a top structure, a bottom structure, and a substrate. In the exemplary embodiment provided in FIG. 13, the substrate and electronics are located at the base of the sensor. The bottom structure surrounds the perimeter of the substrate and has a bar 1315 which cuts across the substrate and fits into an opening in the top structure 1325. The top structure 1310 may be made of a soft material. A non-limiting example of a soft material includes silicone. Further non-limiting soft materials include natural rubber, elastomers and nitrile rubber. The structure 1310 may be in direct contact of the patient body and thus, the soft material may allow long term use of the patch and provide a positive pressure on the patient body. In particular, such a material may allow for comfortable long-term wear, positive pressure on the body as required for physiological readings. The bottom structure 1320 may be made of a hard material. Exemplary hard materials include acrylonitrile butadiene styrene (ABS), polycarbonates (PC), or a combination thereof. The hard material may provide better adhesion to the substrate 1330 on which the electronic and optical components of the sensor are positioned. The hard material may also prevent leakage of light between the electronic components without being reflected through the body layers, when the patient wears the patch, which reduces interference. This may advantageously improve accuracy of data read from the sensors.

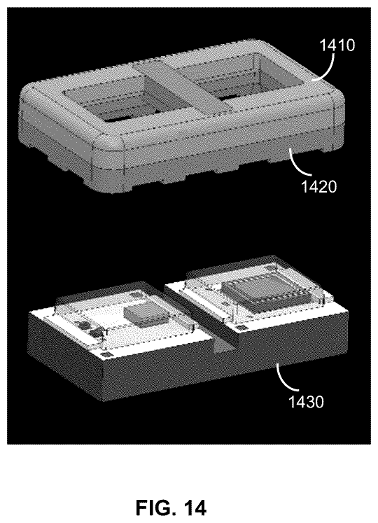

FIG. 14 shows another non-limiting example of a SpO2 sensor, in accordance with some embodiments. The top structure 1410 may be made of a soft material, whereas the bottom structure 1420 may be made of a hard material, as is further discussed in connected with FIG. 13. A non-limiting example of soft material may comprise silicone. Some non-limiting examples of hard material may comprise acrylonitrile butadiene styrene (ABS), polycarbonates (PC), and combination thereof. In some embodiments, the top structure 1410 and/or bottom structure 1420 may be formed by double injection molding.

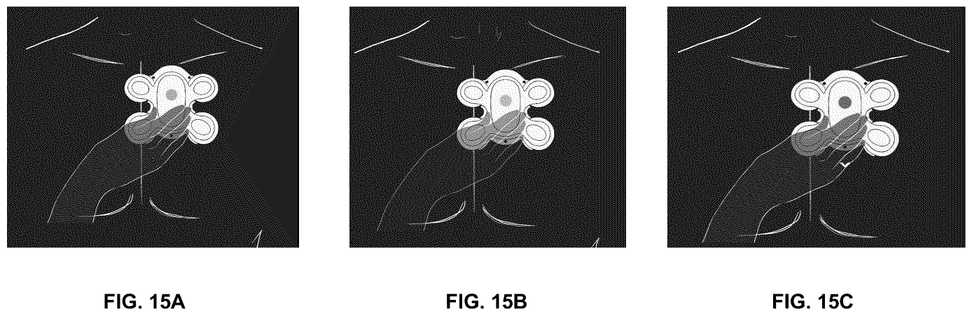

FIGS. 15A, 15B, and 15C show visual indications of a multi-parameter wearable patch, in accordance with some embodiments. The wearable patch may provide visual indications for patch placement confirmation. As illustrated in FIG. 15A, for example, the visual indicator (e.g., LED light) may show orange-colored blinking or flashing amber, indicating the patch is assessing whether its current position on the body of the patient is good. When the patch is applied to a good position, the visual indicator may show a stable green color (see, e.g., FIG. 15B). When the position is not good, the visual indicator may show a stable red color (see, e.g., FIG. 15C). The patient may be asked to move the patch to a different location until the visual indicator shows a stable green color.

FIGS. 16A and 16B show non-limiting examples of a multi-parameter wearable patch, in accordance with some embodiments. The wearable patch comprises an electronic module encapsulated with a case. This case is at the top layer of the patch and is made of stretchable material 1610. The case may contain a central portion with extensions. For example, FIG. 16A provides a central, raised rectangular portion with four walls projecting from the base of the patch, and having four extensions at each corner of the base (i.e., from the ring island electrodes). The bellow design of the case, with its floating rigid center connected to flexible extensions, avoids lift off of the sensor which otherwise would result from the contour of the sternum or left or right chest abdomen. The electronic module or case is located on top of a layer of stretchable material, providing support for the structure while also maintaining optimal flexibility when the patch is applied to body contours, improve sensor to skin contact, reduce shear stress, and avoid peel off or lift off of the patch. Hence, the lifetime of the patch as well as the quality and reliability of collected physiological signals may be increased.

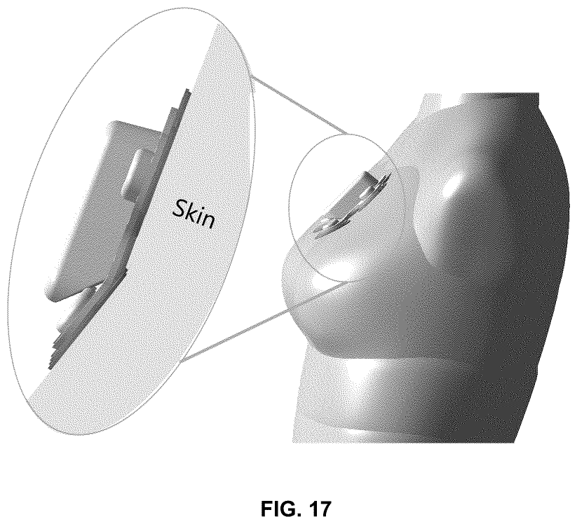

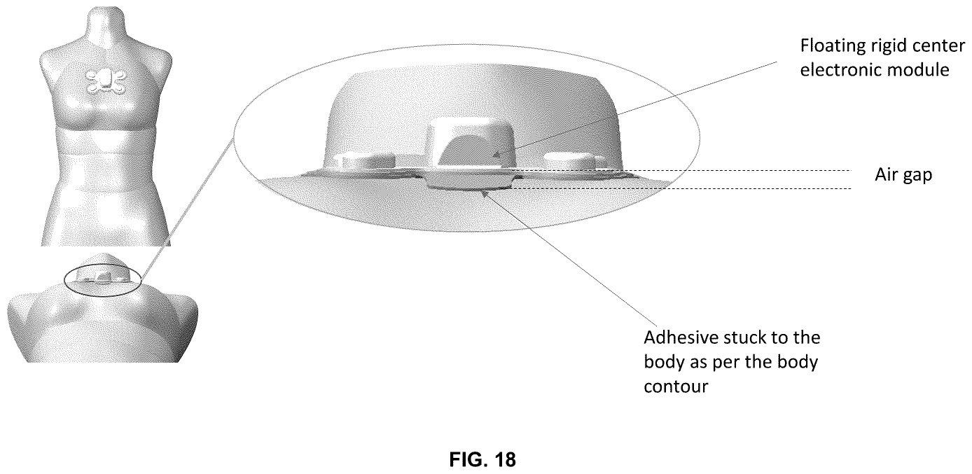

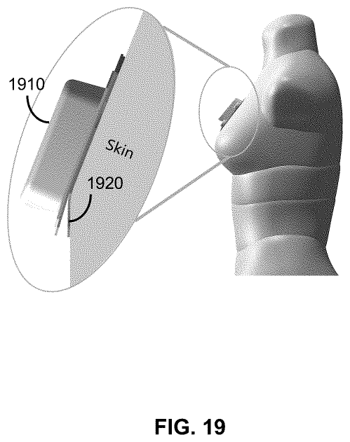

FIGS. 17-19 show non-limiting examples of a multi-parameter wearable patch placed on the chest of a subject, in accordance with some embodiments. As described above, the wearable patch may have combined structures made of different materials, including but are not limited to, island ring electrode design as illustrated in FIGS. 5 and 6; adhesive and non-adhesive regions as illustrated in FIGS. 7-11; and optical sensors made of hard and soft materials as illustrated in FIGS. 12-14. These structures and materials may allow the patch to better fit the contour of the patient body, for example, left or right chest of female patients or muscular male patients. FIG. 17 provides the wearable patch on the skin of the subject. The patch in FIG. 17 is configured to fit on the chest of a female subject. FIG. 18 and FIG. 19 provide additional views of this placement on a female.

As illustrated in FIG. 17 and FIG. 18, the patch may have a floating rigid center where an electronic module of the patch is placed. The patch may also have an adhesive layer attached to the patient body along with contour. In FIG. 17, the skin contact adhesive layer can be seen in contact with the skin of the patient. In FIG. 18, an air gap is visible between the skin contact adhesive layer and the rigid, central portion of the patch. This is further illustrated in FIG. 19, which shows an air gap between the adhesive layer 1920 and floating rigid center 1910. Such design may allow the patch to better fit the contour of the patient body, for example, sternum (central chest) of female patients or muscular male patients because the flexible portion is in contact with the skin while the more rigid portion is floating above, but still connected to the flexible portion at certain location (such as near the upper part of the patch in FIG. 19).

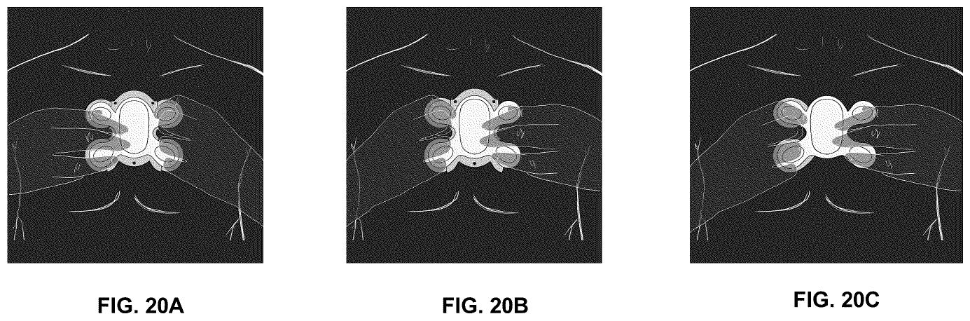

FIGS. 20A, 20B, and 20C show placement of a multi-parameter wearable patch comprising one or more release liners, in accordance with some embodiments. As illustrated in FIG. 20A, the patch comprises one or more removable release liners, which make up separate portions of a single layer of release liners. A subject may identify a suitable position on the body to place the patch without removing the release liners. Once the suitable position is identified, the patient may remove each release liner in sequence, expose the adhesive layer of the patch to the body (see, e.g., FIG. 20B where the position has been identified and the liner at side portion of the liner is removed, and FIG. 20C where the adhesive layer has been exposed, with one side release liner and the central release liner removed, and one final release liner remaining). During the removal process of each release liner, some portion of the patch may always be held on the patient body, thereby ensuring previously identified suitable positions maintained and unaltered.

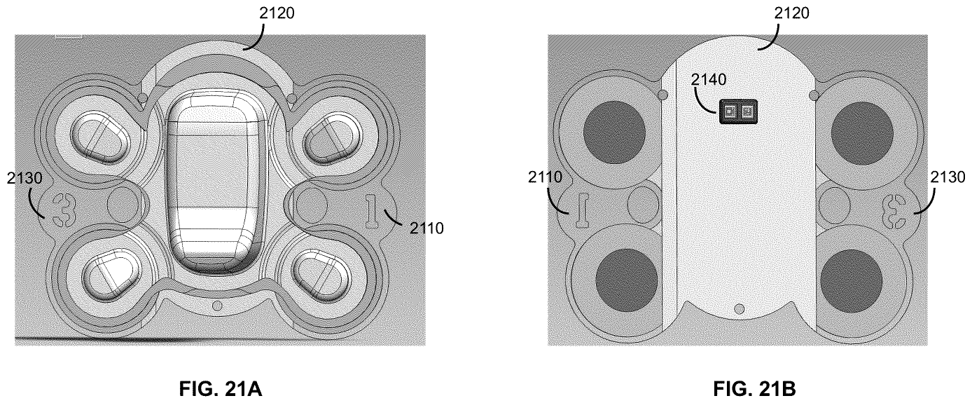

FIGS. 21A and 21B show non-limiting examples of a multi-parameter wearable patch release liners, comprising combinations of paper release liners and plastic release liners, in accordance with some embodiments. The liners can be separated into multiple portions, each of which has a separate release liner layer. In FIG. 21A and FIG. 21B, three separate release liner portions are shown, two of which have stencil markings (e.g., the stencil for “1” at the first release liner 2210 and the stencil for “3” at the side release liner 2130), and one which is located at the central portion of the patch (2120). In the exemplary configurations provided in FIG. 21A and FIG. 21B, the release liners comprise central portions with paper release liners 2120 and outer portions with plastic release liners (e.g., 2110 and 2130). In some embodiments, the plastic release liners may be positioned on the center of the skin adhesive layer, and paper release liners may be placed on both sides. In other embodiments, the paper release liner may be positioned on the skin adhesive layer at the central portion of the patch (e.g., 2120), and the plastic release liners may be placed on both sides due to hydrogel (e.g., at 2110, 2130), which is shown in FIG. 21A and FIG. 21B.

As illustrated, the release liners may also have stencil and/or printed marks (e.g., numbers 1-3 or alphabet A-C) for ease of identification of an order in which the release liners are to be removed in sequence. For example, in both FIG. 21A and FIG. 21B, a ‘1’ and ‘3’ stencil are located on the sides of the stencil, between two extensions where two island ring electrodes would be located relative to the patch (see, e.g., 2110 and 2130 in FIG. 21B). As illustrated in FIG. 21B, the release liners may also have cuts or openings for an optical sensor 2140 (e.g., SpO2 sensor), such that when the patch is adhered to a suitable location on the body, the optical sensor may function properly.

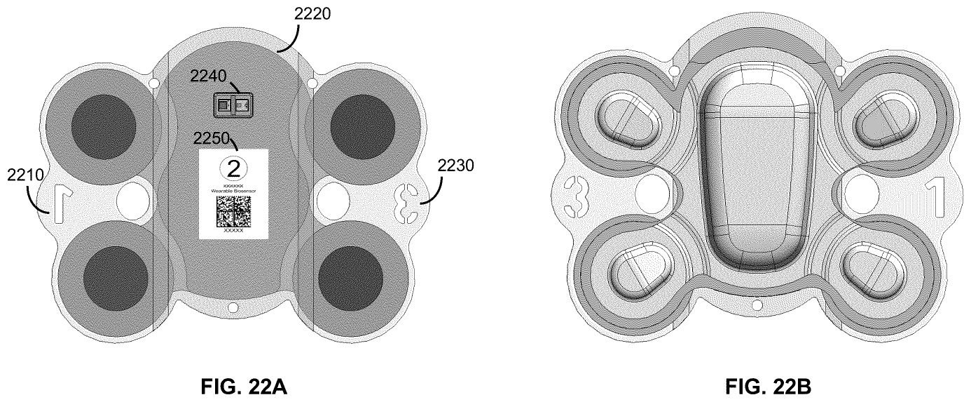

FIGS. 22A and 22B show non-limiting examples of plastic release liners for patches provided herein, in accordance with some embodiments. As illustrated, the release liners (e.g., 2210, 2220, and 2230) may also have cuts or openings for an optical sensor 2240 (e.g., SpO2 sensor), such that when the patch is adhered to a suitable location on the body, the optical sensor may function properly. The release liners may also have stencil and/or printed marks (e.g., numbers 1-3 or alphabet A-C) for ease of identification of an order in which the release liners are to be removed in sequence. For example, FIG. 22A shows a plastic release liner with a plastic central portion (2220), a plastic left portion (2210) and a plastic right portion (2230). The right portion 2230 has a stencil mark of a ‘3’ and the left portion 2210 has a stencil mark of a ‘1’. A ‘2’ is printed (2250) on the center of the release liner. The cut for the optical sensor 2240 is located in the central portion of the release liner. The optical sensor projects from this portion of the patch to provide positive pressure on the body, which assists in sensor readings (e.g., for chest-based SpO2 readings). The cut allows for removal of the release liner without disturbing the position of the sensor.

### Computer Systems

In some examples, the platforms, systems, media, and methods described herein may include a digital processing device, or use of the same. Any computing device described herein may be a digital processing device. In some examples, the digital processing device may include one or more hardware central processing units (CPUs) or general purpose graphics processing units (GPGPUs) that carry out the device's functions. In some examples, the digital processing device may further comprise an operating system configured to perform executable instructions. The digital processing device may be optionally connected a computer network. The digital processing device may be optionally connected to the Internet such that it accesses the World Wide Web. The digital processing device may be optionally connected to a cloud computing infrastructure. The digital processing device may be optionally connected to an intranet. The digital processing device may be optionally connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Many smartphones may be suitable for use in the system described herein. Televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some examples, the operating system may be provided by cloud computing. Suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems may include, by way of non-limiting examples, Apple TV®, Roku®, Boxee®, Google TV®, Google Chromecast®, Amazon Fire®, and Samsung® HomeSync®. Suitable video game console operating systems may include, by way of non-limiting examples, Sony® PS3®, Sony® PS4®, Microsoft® Xbox 360®, Microsoft Xbox One, Nintendo® Wii®, Nintendo® Wii U®, and Ouya®.

The device may include a storage and/or memory device. The storage and/or memory device may be one or more physical apparatuses used to store data or programs on a temporary or permanent basis. The device may be volatile memory and may require power to maintain stored information. The device may be non-volatile memory and retains stored information when the digital processing device is not powered. The non-volatile memory may comprise flash memory, dynamic random-access memory (DRAM), ferroelectric random access memory (FRAM), phase-change random access memory (PRAM).

The digital processing device may include a display to send visual information to a user. The display may be a cathode ray tube (CRT), a liquid crystal display (LCD), a thin film transistor liquid crystal display (TFT-LCD), an organic light emitting diode (OLED) display, a passive-matrix OLED (PMOLED) or active-matrix OLED (AMOLED) display, a plasma display, and/or a video projector.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. The input device may be a Kinect, Leap Motion, or the like. The input device may be a combination of devices such as those disclosed herein.

The platforms, systems, media, and methods disclosed herein may include one or more non-transitory computer readable storage media encoded with a program including instructions executable by the operating system of an optionally networked digital processing device. A computer readable storage medium may be a tangible component of a digital processing device. A computer readable storage medium is optionally removable from a digital processing device. A computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the platforms, systems, media, and methods disclosed herein may include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

A computer program may include a web application. In light of the disclosure provided herein, a web application may utilize one or more software frameworks and one or more database systems. A web application may be created upon a software framework such as Microsoft® .NET or Ruby on Rails (RoR). A web application may utilize one or more database systems including, by way of non-limiting examples, relational, non-relational, object oriented, associative, and XML database systems. In further embodiments, suitable relational database systems include, by way of non-limiting examples, Microsoft® SQL Server, my SQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL).

A computer program may include a mobile application provided to a mobile digital processing device. The mobile application may be provided to a mobile digital processing device at the time it is manufactured. The mobile application may be provided to a mobile digital processing device via the computer network described herein.

A mobile application may be created, for example, using hardware, languages, and development environments. Mobile applications may be written in various programming languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

A computer program may include a standalone application, which is a program that is run as an independent computer process, not an add-on to an existing process, e.g., not a plug-in. Standalone applications may be compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program.

The computer program may include a web browser plug-in. In computing, a plug-in may be one or more software components that add specific functionality to a larger software application. Makers of software applications support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Web browser plug-ins include, without limitation, Adobe® Flash® Player, Microsoft® Silverlight®, and Apple® QuickTime®. The toolbar may comprise one or more web browser extensions, add-ins, or add-ons. In some embodiments, the toolbar comprises one or more explorer bars, tool bands, or desk bands.

Several plug-in frameworks may be available that may enable development of plug-ins in various programming languages, including, by way of non-limiting examples, C++, Delphi, Java™, PHP, Python™, and VB.NET, or combinations thereof.

Web browsers (also called Internet browsers) are software applications, which may be configured for use with network-connected digital processing devices, for retrieving, presenting, and traversing information resources on the World Wide Web. Suitable web browsers include, by way of non-limiting examples, Microsoft® Internet Explorer®, Mozilla Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) may be configured for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

The systems, media, networks and methods described herein may include software, server, and/or database modules, or use of the same. Software modules may be created using various machines, software, and programming languages. The software modules disclosed herein are implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. The one or more software modules may comprise, by way of non-limiting examples, a web application, a mobile application, and a standalone application. In some embodiments, software modules are in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The platforms, systems, media, and methods disclosed herein may include one or more databases, or use of the same. In view of the disclosure provided herein, many databases are suitable for storage and retrieval of physiological data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. A database may be web-based. A database may be cloud computing-based. A database may be based on one or more local computer storage devices.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A patch for monitoring physiological data comprising:
(a) a base configured to come in contact with a surface of a user;
(b) one or more sensors operably coupled to the base, the one or more sensors configured to monitor the physiological data from the user; and
(c) an electronic module in communication with the one or more sensors, the electronic module configured to receive the monitored physiological data,
wherein the base comprises one or more adhesive regions and one or more non-adhesive regions, and the one or more non-adhesive regions are configured to flatten or bulge between the adhesive regions,
wherein the sensors comprise SpO2 sensors, and each of the SpO2 sensors comprises a top structure, a bottom structure, and a substrate positioned therebetween.

2. The patch of claim 1, wherein the top structure comprises a soft material, and the bottom structure comprises a hard material.

3. The patch of claim 2, wherein the soft material is silicone.

4. The patch of claim 2, wherein the hard material is acrylonitrile butadiene styrene (ABS), polycarbonates (PC), or a combination thereof.

5. The patch of claim 3, wherein the hard material is acrylonitrile butadiene styrene (ABS), polycarbonates (PC), or a combination thereof.

6. The patch of claim 1, wherein the top structure and the bottom structure are injection-molded.

7. The patch of claim 2, wherein the top structure and the bottom structure are injection-molded.

8. The patch of claim 3, wherein the top structure and the bottom structure are injection-molded.

9. The patch of claim 4, wherein the top structure and the bottom structure are injection-molded.

10. The patch of claim 5, wherein the top structure and the bottom structure are injection-molded.

11. A method of monitoring physiological data of a user, the method comprising:
(a) contacting a surface of a user with a patch, wherein the patch comprises:
(1) a base configured to come in contact with a surface of a user;
(2) one or more sensors operably coupled to the base, the one or more sensors configured to monitor the physiological data from the user; and
(3) an electronic module in communication with the one or more sensors,
the electronic module configured to receive the monitored physiological data, wherein the base comprises one or more adhesive regions and one or more non-adhesive regions, and the one or more non-adhesive regions are configured to flatten or bulge between the adhesive regions; and
(b) monitoring physiological data of a user from the patch,
wherein the sensors comprise SpO2 sensors, and each of the SpO2 sensors comprises a top structure, a bottom structure, and a substrate positioned therebetween.

12. The method of claim 11, wherein the top structure comprises a soft material, and the bottom structure comprises a hard material.

13. The method of claim 12, wherein the soft material is silicone.

14. The method of claim 12, wherein the hard material is acrylonitrile butadiene styrene (ABS), polycarbonates (PC), or a combination thereof.

15. The method of claim 13, wherein the hard material is acrylonitrile butadiene styrene (ABS), polycarbonates (PC), or a combination thereof.

16. The method of claim 11, wherein the top structure and the bottom structure are injection-molded.

17. The method of claim 12, wherein the top structure and the bottom structure are injection-molded.

18. The method of claim 13, wherein the top structure and the bottom structure are injection-molded.

19. The method of claim 14, wherein the top structure and the bottom structure are injection-molded.

20. The method of claim 15, wherein the top structure and the bottom structure are injection-molded.
